(19) United States
(12) Patent Application Publication
(10) Pub. No.: US 2023/0381360 A1
(43) Pub. Date: Nov. 30, 2023

(54) DEVICE FOR ACCESS CONTROL WITH PHYSICAL DISINFECTION

(71) Applicant: JK-HOLDING GMBH, Windhagen (DE)

(72) Inventor: Jürgen GERSTENMEIER, Neuwied (DE)

(73) Assignee: JK-HOLDING GMBH, Windhagen (DE)

(21) Appl. No.: 18/250,130

(22) PCT Filed: Oct. 20, 2021
(86) PCT No.: PCT/IB2021/059647

§ 371 Date: Apr. 21, 2023

(30) Foreign Application Priority Data
Oct. 23, 2020 (CH) 01364/20

(51) Int. Cl. A61L 2/24; A61L 2/10; G07C 9/10

(52) U.S. Cl. CPC A61L 2/24; A61L 2/10; A61L 2202/25; A61L 2202/14; A61L 2202/11; G07C 9/10

(57) ABSTRACT

The present invention relates to a device (1) for access control. The device (1) comprises a first physical barrier (1) for delimiting an irradiation space (2) along a passage direction (A). The device further comprises an irradiation device (10) for subjecting a living being (3) in the irradiation space (2) to optical radiation in a wavelength range of between 200 and 230 nm, particularly preferably to optical radiation with a peak in a wavelength range of between 207 and 222 nm. The present invention also relates to a method for access control and to a use of said device.

The present invention relates to a device for access control with an integrated physical disinfectant, as well as a corresponding method for access control, all in accordance with the preambles of the independent claims.

### TECHNICAL BACKGROUND

There is a need for regulated access control in private or public buildings or sites, in which, in addition to the usual controls, a further hygienic safety level is provided. For example, especially sensitive buildings or sites, such as assisted living and nursing homes, may need, in addition to normal access control, or as an alternative to this, to ensure that people entering the site or building have performed a certain degree of disinfection of their hands or other body parts.

In times of heightened pandemic alertness, a container with disinfectant is usually set up in entrance areas, for example in shopping centers, hospitals, or nursing homes. This assumes that every visitor uses the disinfectant conscientiously and correctly. As before, however, dangerous germs may still be present on clothing, shoes, or other parts of the visitor's body and thus get into the site.

In times of heightened pandemic alertness, there is also a need to design access gates for especially sensitive areas in such a way that essentially complete disinfection of all surfaces can take place. Ideally, such a device can be used in a modular manner and installed at short notice if necessary. Of course, such a device can be an integral part of a disinfection gate, such as those set up in corresponding intensive care units, quarantine rooms, or operating theaters in hospitals.

Previous systems are not able to ensure that all persons conscientiously and correctly carry out the necessary hygienic safety measures. There is, therefore, a need for devices for access control that are secure and meet the high level of hygienic requirements for access to a site or building.

### PRESENTATION OF THE INVENTION

It is, therefore, an object of the present invention to provide a device for access control which overcomes at least one disadvantage of the known devices. In particular, a device for access control is to be provided which guarantees a standardized requirement for disinfection for the corresponding access control. The device for access control can preferably be networked with other systems.

At least one of these objects was achieved with a device for access control according to the characterizing part of the independent claims.

One aspect of the present invention relates to an access control device. The device comprises a first physical barrier for restricting access to an irradiation space along a passage direction.

The device further comprises at least one irradiation device for applying optical radiation to a living being in the irradiation space. The optical radiation has a wavelength range of between 200 and 230 nm. The optical radiation especially preferably has a peak in a wavelength range of between 207 and 222 nm, and very especially preferably the peak of the optical radiation is approximately 207 nm or approximately 222 nm, in which “approximately” is to be understood as a peak deviation of between ±2 nm.

Physical disinfection takes place due to exposure of the living being and any clothing worn by the living being and/or objects. The UV-C radiation emitted in said wavelength range is especially suitable for rendering microorganisms harmless, for example by causing DNA and RNA damage in these organisms and thus reducing the pathogen potential of bacteria, viruses, fungi, and other possible pathogens. Said wavelength range is largely harmless to higher life forms (cf. Long-term effects of 222 nm ultraviolet radiation C sterilizing lamps on mice susceptible to ultraviolet radiation, Yamano, Nozomi et al., Photochemistry and Photobiology, doi: 10.1111/php.13269).

One advantage of the mentioned device for access control is that this radiation, which is harmless to humans, can be set up in a public space and operated continuously. In this way, regardless of conscientiousness when disinfecting, for example the hands, it can be ensured that a minimum of disinfection has been performed on a person seeking access to a building or site. The irradiation device is especially preferably designed to inactivate viruses from the corona virus family and to emit UV radiation with a peak in a wavelength range of between 207 and 222 nm, with an energy of between 0.3 mJ/cm² and 500 mJ/m² in the irradiation space, in particular of between 2 mJ/cm² and 50 mJ/cm², very especially preferably of approx. between 2 mJ/cm² and 20 mJ/cm².

Without being bound by this theory, the wavelength ranges mentioned seem to be wavelengths that are mainly absorbed in the skin surface, the cuticle, and which do not succeed in penetrating human cells and causing undesired cell damage there, as can occur with other UV radiation. In the context of the present invention, the passage direction can be defined on one side or on both sides. For example, a device according to the invention can also be provided in order to carry out the appropriate disinfection only after a building or site is exited. A device according to the invention can, for example, also be designed to be passable in only one direction. Exiting of the building or site would then take place, for example, via a second device which is arranged in the direction opposite the passage direction and thus separates a passage flow from the entrance flow.

In a particular embodiment, the irradiation space is defined in such a way that at least parts of the body of the living being are covered by the optical radiation. For example, the irradiation space can be designed to capture at least the hands of the living being.

In a particular embodiment, the device has a modular structure, so that a module is designed to include an irradiation space for at least parts of the body. For example, the irradiation space can be designed to capture at least the hands of the living being. Individual modules can be designed so that they can be combined to capture an entire living being in an irradiation space. This can be advantageous, for example, when individual parts of a body require different doses of energy in order to be adequately disinfected. It can also be advantageous if objects that are carried are also to be subject to disinfection. For example, an example of a module can serve as a collecting container for objects carried in the hands. Thus, a gate according to the invention can also be designed with a module for the hands and a storage module. Thus, both a corresponding irradiation space for the hands and one for the deposited objects can be provided, which enables especially safe disinfection.

In the context of the present invention, living beings can be people who are seeking access to a building or site, for example. However, it is also conceivable to use the device mentioned in an agronomic operation, in which case the living beings mentioned can be animals. Accordingly, an animal-friendly disinfection can be carried out at certain gates with the device according to the invention. Coupled with other functions, the operation of such a gate is especially advantageous because a fully automatic process can be set up that ensures that certain areas which animals can independently access are exposed to a comparatively lower bacterial load when the animals pass through the corresponding device for access control.

In a particular embodiment, the irradiation device comprises a lighting means that is excimer-based. This is especially preferably a Kr—Br-excimer lamp or a Kr—Cl lamp. Excimer lamps are used in many industrial applications and work on the basis of an excited dimer (e.g., Kr—Cl gas) in that an alternating current is applied and this dimer is put into a higher energy state. Synthetic quartz glass creates a physical barrier between at least one electrode. Well-known areas of application for excimer lamps include semiconductor production, in which wavelengths with peaks in the range of 172 nm are used to break down organic compounds and generate ozone to combat dirt particles.

In a particular embodiment, the lighting means is an excimer-based lamp which essentially emits light of a wavelength with a peak of 207 nm, in particular a wavelength with a peak of essentially 207 nm, in which the emission spectrum is >than 200 nm and <than 214 nm, especially preferably >than 204 nm and <than 210 nm, at a relative power of ten percent or more.

In an alternative particular embodiment, the lighting means comprises an excimer-based lamp which essentially emits light of a wavelength with a peak of 222 nm, in particular a wavelength with a peak of essentially 222 nm, in which the emission spectrum is >than 215 nm and <than 229 nm, especially preferably >than 219 nm and <than 225 nm, at a relative power of ten percent or more.

In a particular embodiment, a device according to the invention can have a plurality of irradiation devices, wherein each irradiation device can have a different excimer-based lighting means.

In addition to the corresponding pair of dimers, a lamp according to the invention can comprise a suitable shortpass and/or bandpass filter. In a further particular embodiment, the shortpass filter has an interference filter made up of at least one, preferably two, filter layers.

In a particular embodiment, the device according to the invention comprises a first sensor. The first sensor is especially preferably an optical sensor. For the purposes of the present invention, an optical sensor is primarily suitable for detecting visible or invisible light, for example. Such a sensor can, for example, also be an infrared sensor which is able to detect infrared light.

In a further particular embodiment, the optical sensor is additionally an image sensor which is able to record light in an image.

The image sensor is especially preferably designed to record images in the infrared range.
In an especially preferred embodiment, the first sensor comprises a focal plane array. This sensor is designed to place a row of optical sensors in an arrangement.

In a particular embodiment, the first sensor is an infrared sensor which is designed to record a thermal image of a living being in the irradiation space.

In a particular embodiment, the physical barrier can be converted from a closed to an open state. In the context of the present invention, a physical barrier can be understood as a barrier that prevents a living being from continuing in a passage direction directly, i.e. by blocking for example, or indirectly, by means of instructions for example. In a particular embodiment, such a physical barrier would sensibly be attached to an entrance to a building or site. The physical barrier can comprise, for example, an effective barrier, such as a glass door, a tree, a portal, a sliding door, or a pivoting door; however, it can also be realized by means of a directly recognizable instruction not to go any further, which is recognized by the living being. For example, simple traffic lights with a red-green system can be sufficient as a physical barrier to delimit an irradiation space.

In the context of the present invention, the closed state of a physical barrier can be understood as the state in which the living being is not prevented from advancing in the passage direction and no corresponding instructions prevent the living being from continuing in this passage direction. Correspondingly and analogously, the open state would enable the living being to continue in the passage direction, or no optical or auditory signals would try to prevent the living being from doing so. A barrier can be converted in that it can transition from an open or closed state to the other respective state, for example by fulfilling a predefined condition. In a particular embodiment, a certain length of stay in the irradiation space can be provided as a predefined condition.

In a particular embodiment, the device according to the invention comprises a control unit for actuating the physical barrier. The control unit is designed to actuate the physical barrier on the basis of predefined criteria. This actuation can include, for example, a predefined criterion from the group consisting of: length of stay of the living being in the irradiation space, body temperature of the living being, change in the body temperature of the living being, exposure time of the living being to optical radiation in a wavelength range between 200 nm and 230 nm, exposure intensity of the living being to optical radiation in a wavelength range between 200 nm and 230 nm, changes in the surface temperature of the living being, the medical condition of the living being, and optical recognition of the living being.

In a particular embodiment, the first sensor is designed to detect, measure, or record at least one of these predefined criteria on the living being. In a specific example, the first sensor could be an infrared sensor which is designed to record a thermal image of a living being in the irradiation space. A predefined criterion could be, for example, a body temperature of the living being or, for example, a change in the surface temperature of the living being. In this example, the control unit could be designed to actuate the physical barrier, that is to say, for example, to transition it from a closed to an open state when a certain change in the surface temperature of the living being is detected by the first sensor. It is thus possible to determine whether and to what extent the irradiation device has sufficiently captured the living being, and thus sufficient disinfection of the surfaces of the living being has taken place. It goes without saying that not only the surfaces on the skin are adequately disinfected by this irradiation device, but also corresponding surfaces on clothing and/or, if need be, objects carried by the living being in the hands or on the back.

In a particular embodiment, corresponding protective devices which the living being wears on the body are also sufficiently irradiated by the irradiation device. A change in the surface temperature of a protective suit can serve as an indication that this protective suit has already been sufficiently irradiated. Any folds or kinks or shading of the protective suit which prevent the protective suit from being completely irradiated are identified through corresponding detection by means of the infrared sensor. In this example, auditory or optical information could then also be transferred to the living being, which makes it possible to specifically expose the correspondingly shaded areas so that a comprehensive disinfection can take place. In the context of the present invention, an irradiation space can be seen as a correspondingly defined spatial region in which the irradiation device is able to apply the optical radiation with a desired intensity. Correspondingly, the irradiation space can be defined in close proximity to the physical barrier. The irradiation area can be understood either as a defined space, i.e. with physical delimitation, or as a symbolically defined space. Thus, in a specific exemplary embodiment, the irradiation space can be defined by a corresponding marking that instructs a living being to position themselves correctly with respect to the irradiation device. The irradiation space can also be designed to only apply irradiation to parts of the living being. For example, the irradiation space can comprise a compartment, in the interior of which the hands are to be placed and exposed accordingly.

In a particular embodiment, the irradiation space is designed as an irradiation chamber. The physical barrier is designed to essentially hermetically seal off the irradiation space. For example, curtains can be provided which, in a closed state, seal off the irradiation space in a substantially airtight manner.

In a special embodiment, vents can also be provided which generate an overpressure in the irradiation chamber and thus prevent air from entering the irradiation chamber from the outside when it is in a hermetically sealed state. In this way it can be ensured, for example, that a decontamination of a living being, i.e. a disinfection process, is not impaired by germs that have already re-entered from the outside. The disinfection chamber can be made of statically stable materials such as Plexiglas, glass, PVC, or Polydur walls. However, it can also be formed from flexible materials assembled on-site. For example, the disinfection chamber can consist of a framework over which appropriate films are placed, which define the disinfection chamber. The disinfection chamber can correspondingly be hermetically sealed through appropriate vents, as described above.

In particular embodiments, the disinfection chamber is set up as a gate which comprises two physical barriers. A first physical barrier is opened for entry to the disinfection chamber. A second physical barrier is still closed at this time and delimits the irradiation space along the passage direction. The first physical barrier is then closed. The disinfection chamber is hermetically sealed. For this purpose, for example, a gas exchange can also take place in the disinfection chamber. Appropriate vents and/or air-conditioning systems are known to one skilled in the art for ventilating such gate chambers. During this time or afterwards, the disinfection chamber can, as mentioned above, be exposed to the appropriate wavelength
by means of an irradiation device. After certain criteria have been met, the second physical barrier can be opened, and the living being can continue in the passage direction.

In a particular embodiment, the device according to the invention comprises a ventilation unit for conveying an air flow into and/or out of the irradiation space. As already mentioned, such a ventilation flow can be used to convey cleaned air into the irradiation space, for example. Alternatively, this air flow can also be used to evacuate the irradiation space, designed as a disinfection chamber in this specific example.

In a particular embodiment, the ventilation unit comprises a disinfection chamber which is designed to physically disinfect the air flow. The disinfection chamber can comprise, for example, a UV-C lamp which is suitable for essentially disinfecting an air flow as a function of a dwell time of the air flow in an area where the UV-C lamp is applied. Such UV-C disinfection chambers are known in the prior art. In contrast to the UV-C radiation used in the device in the irradiation space, a common UV-C lamp with a wavelength range and a peak of around 254 nm can be used for a disinfection chamber. This wavelength range is a proven range for rendering germs essentially harmless and is used in UV clarifiers for ventilation and water treatment.

In a particular embodiment, the device according to the invention comprises a physical barrier designed as a sliding door. The sliding door can be electronically actuated, for example, and converted from an open to a closed state and back again along guide rails or a slide bearing. A corresponding belt or chain drive can transition the sliding door from one state to the other.

In a particular embodiment, the device according to the invention comprises an emergency release for mechanically transitioning the physical barrier into an open state. Because the emergency release can take place mechanically, it is largely independent of any errors in the operating system of the device and can be carried out by the living being concerned if, for example, the physical barrier does not release after a maximum length of stay in the irradiation space.

In a particular embodiment, the device according to the invention comprises a second sensor for the optical detection of physiognomic properties for the purpose of face recognition. The device according to the invention can be used, for example, as an entry control in a building. Such buildings can, for example, replace a key system in that facial recognition takes place and only authorized persons can enter the building. The device according to the invention thus not only enables control of the access to the building, but also ensures that all the living beings concerned have passed through a predefined disinfection step by staying in the irradiation space for a predefined period of time. Optical face recognition sensors are known. Simple cameras can serve as sensors. The face recognition can take place on a control unit, which compares the corresponding corner points of a vectorized image with a database.

In a particular embodiment, the device according to the invention comprises a third sensor for detecting a living being along a passage direction in front of the device. For example, a step plate or a light barrier can be provided which determines when a living being is moving in an exposure area of the device according to the invention.

In a particular embodiment, this third sensor can also be designed to detect a living being in the irradiation space. Accordingly, the control unit can be designed to initiate a corresponding access program as soon as a living being has been appropriately detected. This access program can contain various predefined processes that regulate the access of the living being to the building or site.

Alternatively, and/or additionally, the third sensor can be designed by means of an infrared sensor in order to detect a temperature change in an irradiation space and thus to enable a control unit to detect the presence of a living being.

In a particular embodiment, the third sensor comprises means for detecting a specific living being. For this purpose, the sensor can be designed to record certain biometric data. This can include face recognition as described at the beginning or corresponding means for capturing unambiguous biometric data, such as fingerprints and/or a human retina. Suitable sensors would be, for example, infrared lasers that operate in a wavelength range between 800 and 900 nm. Most biometric sensors create an image, which in turn is converted into corresponding voxels and compared with a database result.

In a particular embodiment, the device according to the invention also comprises a network connection in order to exchange information with a computer system, such as a server, in a wired or wireless manner.

In a particular embodiment, the electronic components of the device according to the invention are housed in a protected manner. This can mean, for example, that the electronic components are arranged in such a way that they cannot be manipulated by a person who intends to pass through the device in the passage direction without the device being extensively damaged in the process. Corresponding systems are known among experts and can be found in the field of security doors by an interested person skilled in the art.

In a particular embodiment, the device according to the invention comprises an input unit which is suitable for receiving an input from a living being that intends to pass through the device in the passage direction. The input unit can be, for example, a touch-sensitive screen on which a code can be entered accordingly. Such systems are especially suitable if the device according to the invention is to be used, for example, as security for buildings, such as residential or office complexes, and if it is to be ensured that only authorized persons who are in possession of an access code are able to pass through the device according to the invention.

In a particular embodiment, the physical barrier is designed both to grant access to the irradiation space and to enable exiting the irradiation space. For example, a rotatable physical barrier can be set up in such a way that one passage direction always remains open. Such a rotary gate is known in the technical world and can be improved with the teaching according to the invention in that the physical barrier is additionally coupled to a disinfection step, which is ensured by the application of said optical radiation in the irradiction space.

In a particular embodiment, the device according to the invention comprises a control panel for controlling a control unit. This control panel can be necessary, for example, when third parties want to control the device according to the invention. This can be the case, on the one hand, in order to define the corresponding predefined criteria or, for example, when the device for access control is operated by third-party personnel during use. For example, airport staff can directly control a device according to the invention, verify the corresponding identification of the living being in the irradiation space, and at the same time ensure the compliance of the living being, i.e. in the present case the person, with any instructions in the irradiation space in order to carry out the exposure completely.

In a particular embodiment, the irradiation device is arranged to be movable, so that a radiation area can be traversed. In this embodiment, for example, a rail system could be designed in such a way that the irradiation device can be moved along the rail and thus essentially irradiates an irradiation space from all sides. This movement can be controlled by the control unit and take place as a function of these predefined criteria. For example, the speed of the irradiation device can be specified. Corresponding pauses and intervals in the irradiation can also be defined in order to capture parts of the body that are otherwise especially difficult to reach. This movement can, for example, be coupled with further instructions to the living being, i.e. the person for example, in that certain postures are adopted that are intended to ensure that the irradiation device provides sufficient exposure to said optical radiation in largely all surfaces.

A device of this construction can also be stowed in a space-saving manner, and the device could, for example, be set up as movable if required, for example when setting up field hospitals or mobile quarantine stations or operating theaters.

In a particular embodiment, the device according to the invention is designed as a container which has the corresponding irradiation space in its interior. The container has corresponding irradiation devices on at least two container walls and an entrance area and an exit area. In this case, the exit area assumes the role of a physical barrier, which prevents the living being from advancing in the passage direction. The container can be provided with corresponding connections in order to be coupled to a power supply accordingly. It is also conceivable that the container is equipped with appropriate energy sources that enable it to operate in the field for at least a certain period of time. Corresponding batteries or accumulators, which can be charged, can be provided. The batteries are especially preferably replaceable. It is also conceivable that the corresponding containers are equipped with solar cells, which can be used to charge the energy carrier and to provide energy for operation.

For one skilled in the art, it goes without saying that the features mentioned can be realized in an embodiment according to the invention in any combination, provided they are not mutually exclusive. Furthermore, one skilled in the art understands that the method features mentioned below can also constitute structural features which can be used in an implementation according to the invention of a device for access control.

The solution according to the invention provides a technology which can be used in a variety of ways including to secure fixed installations, such as buildings or sites, to secure certain areas and complexes within buildings, such as intensive care units or operating theaters, as well as flexible and modular use in the field, e.g. for crisis and disaster management.

One aspect of the present invention relates to a method for access control. In the method according to the invention, a device for access control is to be provided, especially preferably a device for access control of the type mentioned at the beginning.

The method according to the invention further comprises the step of transitioning a physical barrier of the device for access control from an open to a closed state as soon as a living being is in an irradiation space of the device for access control. Alternatively, the physical barrier can already be in a closed state when the living being enters the irradiation space.

The irradiation space is subjected to exposure with an irradiation device, the irradiation device being designed to emit optical radiation in a wavelength range of between 200 and 230 nm, in particular optical radiation with a peak in a wavelength range of between 207 and 222 nm.

In a particular embodiment, the method according to the invention comprises the step of detecting at least one living being in the irradiation space by means of a sensor, in particular an optical sensor.

In an embodiment according to the invention, the method comprises the steps of generating a thermal image of the living being before the start of the exposure, in particular by means of an infrared sensor, and further continuous recording of a thermal image of the living being during the exposure.

This method ensures that a corresponding irradiation of the entire surface of the living being has taken place. Without being bound by this theory, there appears to be a positive effect in the optical radiation of the wavelength mentioned in the UV-C range in that it does not cause any of the cell damage usually associated with UV radiation. This is due to the fact that the wavelength ranges mentioned are mainly absorbed on the surface of the skin. This leads to warming of the corresponding skin area, so that a difference, measurable by infrared sensors, can indicate the extent to which irradiation with said radiation was sufficient for inactivating a certain amount of germ-forming organisms and/or viruses.

In a particular embodiment of the method according to the invention, a control unit actuates a transition of the physical barrier from a closed to an open state. This is done using predefined criteria. Especially preferably, the physical barrier is actuated by the control unit by means of at least one predefined criterion from the group consisting of: length of stay of the living being in the irradiation space, body temperature of the living being, changes in body temperature of the living being, exposure time of the living being to optical radiation in a wavelength range between 200 nm and 230 nm, exposure intensity of the living being to optical radiation in a wavelength range between 200 nm and 230 nm, changes in the surface temperature of the living being, the medical condition of the living being, and optical recognition of the living being.

Specifically, in a particular embodiment, a control unit, for example, could be designed to actuate a physical barrier on the basis of a measured change in the surface body temperature of a living being by converting the barrier from a closed to an open state. A difference in the measured surface temperature could be detected, for example, with an infrared sensor or a thermal imaging camera. If uniform illumination, i.e., exposure of the surface of the living being too said radiation, is determined in the corresponding wavelength range, the control unit would evaluate this as an indication of sufficient disinfection of the surface and accordingly control the physical barrier in such a way that the living being can pass through the device in the passage direction. Correspondingly, the physical barrier could be actuated on the basis of the other predefined criteria or on the basis of a combination of such criteria. Since the optical radiation in the specified wavelength range is not visible to the human eye, the infrared camera can be used, for example, to ensure that there are no “shadow areas” that would mean insufficient exposure to disinfecting UV-C radiation.

The mentioned wavelength ranges in which there is a peak with a wavelength of either 207 nm or 222 nm are especially preferred. Such a peak can have a deviation of between 1 and 5 nm at the base. Corresponding edge filters are known for generating such a peak. Another predefined criterion can be the length of stay in the irradiation space. A length of stay in the irradiation space is preferably defined in such a way that a certain proportion of viruses and/or viroids are inactivated in the exposure area of the irradiation device in the irradiation space.

Especially preferably, such a length of stay is defined in such a way that at least 90% of the viruses and/or viroids are inactivated in the exposure area of the irradiation device in the irradiation space. Virus inactivation can be regarded as successfully carried out if, for example, the viruses are no longer infectious, i.e. the viruses in question can no longer infect their target cells. Without being bound by this theory, the UV radiation in the wavelength ranges mentioned seems to cause chemical changes in the structural elements of the viruses and/or viroids, which lead to the loss of infectivity. Inactivation can go as far as complete denaturation and disintegration of the virus or viroid. Because the viruses and/or viroids do not have a protective layer, in contrast to higher organisms, UV-C radiation in the wavelength ranges between 200 and 230 nm, which is not especially harmful for eukaryotic organisms, penetrates directly into the DNA or RNA structures of the particular viruses or viroids and leads to damage, for example through dimerization of the nucleic acids, which switch off the replication capacity of the corresponding pathogens.

In a particular embodiment, the control unit is designed in such a way that a length of stay is determined on the basis of data measured by sensors. In this embodiment, a measurement is used, for example, to determine whether sufficient irradiation has taken place. As already described above using the example of the thermal imaging camera, it can be determined, for example, whether and to what extent a surface of a living being has been exposed to the mentioned UV-C radiation, and predefined parameters can be used to determine whether this is sufficient for inactivating a sufficiently high degree of viruses and/or viroids.

In a particular embodiment, a difference in the surface temperature of the living being is determined, and the length of stay of the living being in the irradiation space, in particular in the exposure area of the irradiation unit, is determined on the basis of this difference.

Another aspect of the present invention relates to the use of an irradiation means, which is designed to emit optical radiation in a wavelength range of between 200 and 230 nm, to act on an irradiation space of a device for access control. In this case, the device comprises a physical barrier for restricting access to an irradiation space along a passage direction.

With the method according to the invention and the use mentioned, a system is provided with which buildings, rooms, or sites can be provided with access controls which, in addition to the usual controls of persons, also enable hygienic conditions to be guaranteed in corresponding structures. For one skilled in the art, further advantageous refinements of the present invention result from the combinations of the exemplary embodiments mentioned, as well as the following detailed, specific embodiments.

The invention will now be explained in more detail in the following on the basis of specific exemplary embodiments and figures without, however, being restricted to these.

The figures are schematic and, for the sake of simplicity, equivalent parts have been given the same reference numerals.

### DESCRIPTION OF FIGURES

The following is shown:

FIG. 1 a schematically a device according to the invention for access control;

FIG. 1b the device of FIG. 1a with additional sensors;

FIG. 2a a further device according to the invention for access control;

FIG. 2b the device of FIG. 2a in a different perspective;

FIG. 3a a further embodiment of a device according to the invention for access control;

FIG. 3b the device according to FIG. 3a in a section through the housing;

FIG. 4 a further device according to the invention for access control;

FIG. 5a a further device according to the invention for access control;

FIG. 5b the figure of the device according to 5a in a view with partial cut-outs;

FIG. 6 schematically the principle of a device according to the invention for access control; and

FIG. 7 a device according to the invention with a movable irradiation device.

### EXECUTION OF THE INVENTION

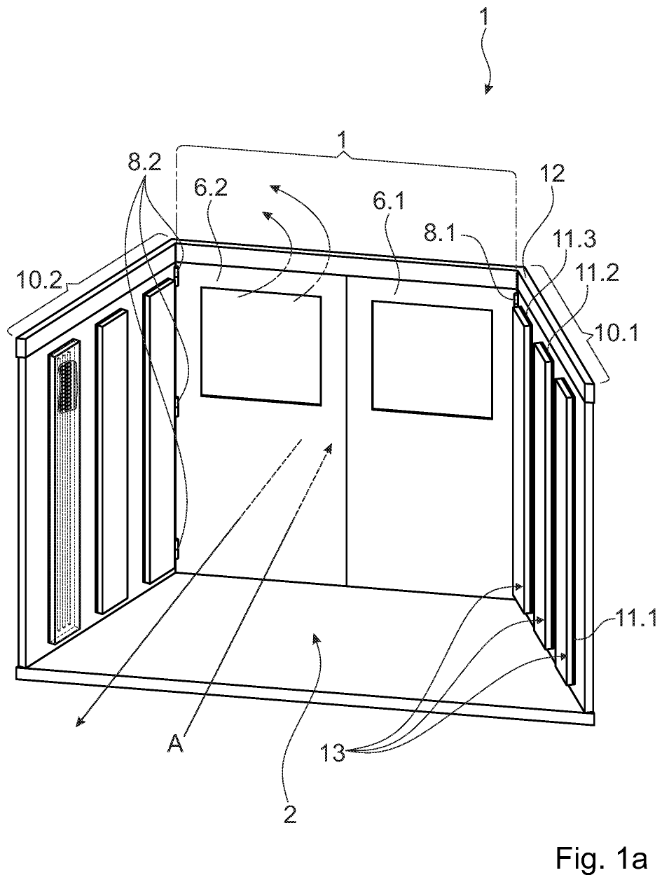

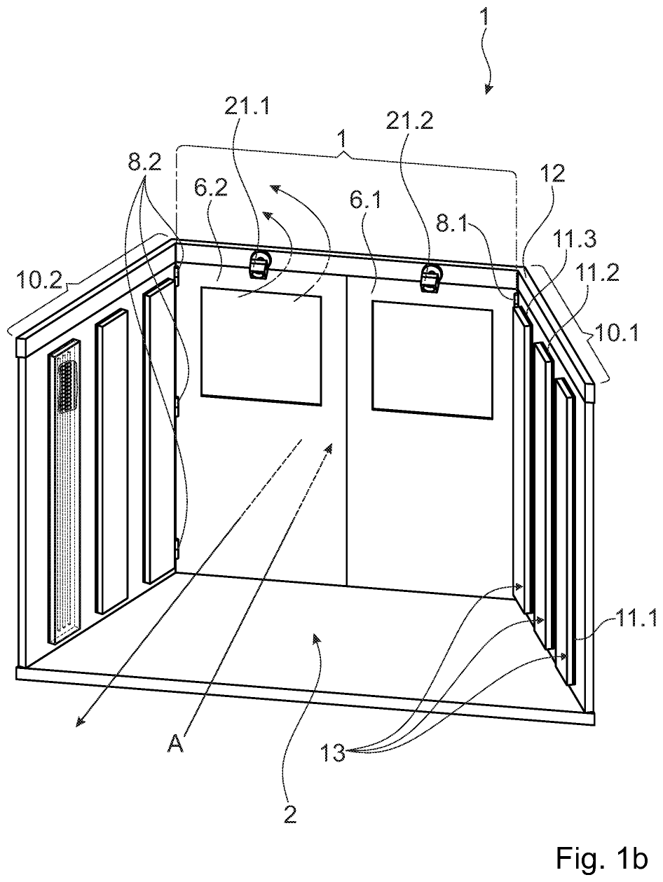

FIG. 1 shows a device 1 according to the invention as it can be used, for example, for access control to buildings or sites. The device 1 is physically blocked in a passage direction A so as to prevent a person from entering the building without undergoing a disinfection process. The device 1 shown by way of example has a substantially trapezoidal structure and defines an irradiation space 2, which is positioned directly in front of the physical barrier 1 in the passage direction A. This irradiation space 2 is selected so that the irradiation devices 10.1, 10.2, which are arranged on both sides of the physical barrier 1 at an approximate 45 degree angle, illuminate it completely. Optionally, further irradiation devices can be accommodated in the floor of the irradiation space 2, for example by covering the floor with a pane of glass. Likewise, pictograms can optionally be provided which instruct persons who would like to pass through the physical barrier 1 in passage direction A how they must stand in order to be optimally aligned with the irradiation devices 10.1, 10.2 and possibly also instruct them to spread apart their arms or hands in the direction of the irradiation devices 10.1, 10.2. In the present example, the physical barrier 1 comprises two swing door halves 6.1, 6.2. These can be converted from a closed state, as shown, into an open state by means of corresponding hinges 8.1, 8.2. To keep this from happening without the person undergoing a disinfection process, a control unit (not shown) can be provided which controls a latch that locks the two swing door halves 6.1, 6.2 and/or blocks the hinges 8.1, 8.2.

The irradiation devices 10.1, 10.2 shown in this example comprise a plurality of lighting means 11.1, 11.2, 11.3, which are each provided with a cover plate 13. The outermost lighting means on the left in the viewing direction is shown by way of example, in which it is possible to see through the glass cover into the interior of the lighting means. A Kr—Br excimer tube was installed as the lighting means. These tubes are installed in series as groups of three in an irradiation device. Heat exchangers and/or ventilation elements can also be provided on the rear sides of the irradiation devices 10.1, 10.2 in order to dissipate the respective heat generated by the excimer lamps (not shown in FIG. 1a).

During operation, a person would then step into the irradiation space 2 and undergo a disinfection process, in which the irradiation devices 10.1, 10.2 apply UV-C radiation in the wavelengths between 200 and 230 nm to the irradiation space 2. These wavelengths have been recognized as largely harmless to higher living beings while still performing the inactivation of bacteria, viruses, viroids, and other potential pathogens expected from UV-C radiation. The application takes place over a predefined period and can be controlled by a control unit. It has been shown that an application of an energy of between 0.5 mJ/cm² and 10 mJ/cm² in the radiation room is sufficient for inactivating more than 90% of the viroids and viruses in the radiation room. In the present example, lighting means 11.1, 11.2, 11.3, which are Kr-Br gas lamps, achieve a wavelength with a peak of 207 nm. Shortpass and/or bandpass filters are installed in the lighting means to keep the corresponding peak as narrow as possible. The Kr-Br lamps used here emit a peak at 207 nm with a half-width of approx. 4 nm.

It has been shown that such lamps are sufficient for significantly increasing the corresponding hygiene standards for access controls in buildings.

The variant embodiment of FIG. 1a shown in FIG. 1b also has a pair of sensors 21.1, 21.2. The sensors 21.1, 21.2 shown are optical sensors. These optical sensors can be used, for example, to detect entry into the irradiation space 2, which is open in the viewing direction. Furthermore, these optical sensors can be used to check the effectiveness of the application of the corresponding wavelength. For example, these optical sensors 21.1, 21.2 can be designed as infrared cameras, which are able to detect any increased body temperature of the person wanting access in advance and also to verify the effectiveness of the radiation by determining whether essentially the entire surface of the person was exposed to the disinfecting radiation. For this purpose, the corresponding sensors 21.1, 21.2 can be in operative connection with the control unit and have a corresponding influence on the control of the physical barrier 1, i.e. the swing doors 6.1, 6.2. The sensors 21.1, 21.2 can also be designed in a certain variant in order to control the intensity of the lighting means 10.1, 10.2 with feedback. In the present example, a frame 12 is provided on the device 1B, which serves, on the one hand, as a support frame for panels with lighting means 10.1, 10.2, and also, on the other hand, as stabilization for the physical barrier 1. Furthermore, the frame 12 can also be used as a mounting base for the optical sensors 21.1, 21.2. The frame can be made of stainless steel.

The swing doors 6.1, 6.2 shown here have viewing windows. These viewing windows can serve as an additional safety measure in that a disinfection process can be observed from the outside. The viewing windows can also serve to facilitate visual identification of a person seeking entry.

In addition to the effectiveness control, the optical sensors 21.1, 21.2 can also be designed to supply the control unit with images which it can use to identify the person. The device shown is preferably provided with a network connection (not shown) and a power connection (not shown), which makes it possible to retrieve the corresponding data from an external server or a cloud-based database, if necessary. Suitable optical sensors 21.1, 21.2 can be thermal imaging cameras which measure radiation in a wavelength range of between 0.5 and 1000 μm. Cameras that are designed to create thermographic images are suitable, for example. The thermographic image can especially preferably also be used to identify a person, for example by vectorization and face recognition. In the present example, a camera with a detector field of (1.024×768) IR pixels, a thermal resolution of 0.02 K, and an IR image frequency of 240 Hz was used.

Optionally, output units, such as loudspeakers for example, can also be provided which give additional auditory instructions to the person seeking entry, for example the positioning for disinfection described at the beginning with reference to FIG. 1a.

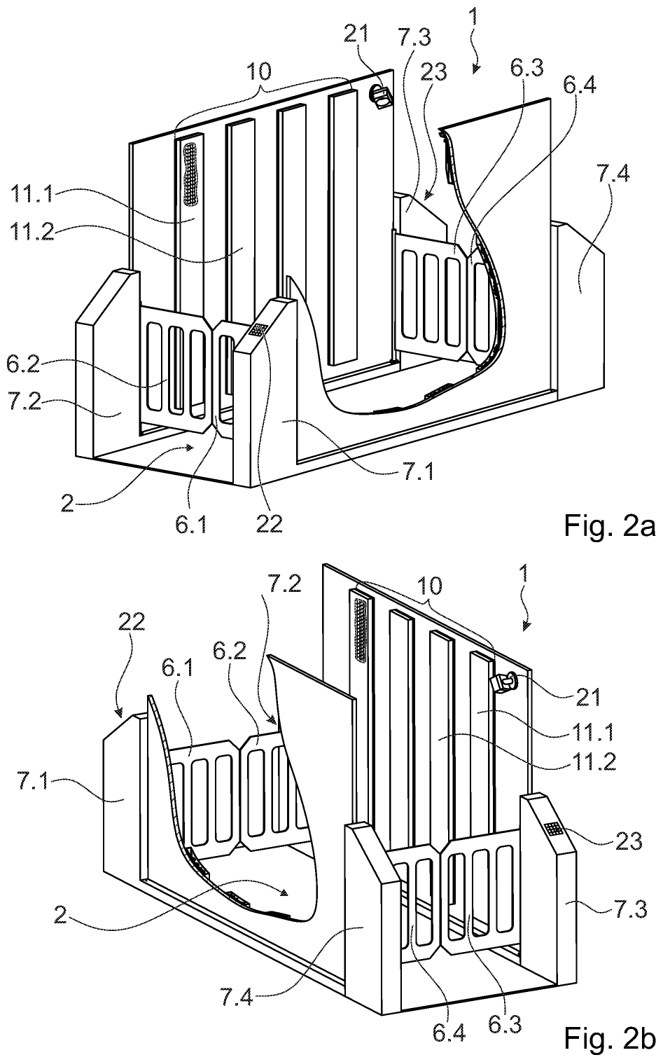

FIG. 2a shows a device 1 according to the invention, in which a defined irradiation space 2 is delimited by two physical barriers and by panels with irradiation devices 10. The device shown in FIG. 2a is constructed like a passage or a gate through which a person seeking entry can pass. In the perspective view, a first physical barrier is shown, which has two swing doors 6.1, 6.2, which are installed at a first control station 7.1 or a second control station 7.2 so as to be opened and closed. An input unit 22, which can be used to pass through this first physical barrier and to enter the irradiation space, is provided at the first control station 7.1. This input unit 22 can also comprise a near-field sensor or a scanner, such as an optical sensor, which is suitable for reading an access card. The operator can use either a code, a release key, or an access card in order to overcome this first physical barrier and access the irradiation space 2. The irradiation space 2 here is an example with an irradiation device 10 on each side of the wall, in which the irradiation device closer to the viewer is shown as a section for better illustration of the interior. The irradiation space 2 is designed here as a corridor through which the person seeking entry must pass. An optical sensor 21 is attached to the opposite end and to the second physical barrier, which allows exit. The second physical barrier can also be equipped with a first station 7.3 and a second station 7.4, which enables the device according to the invention to be operated equally from both sides in this essentially symmetrically arranged system. In this way, a person can walk in a passage direction from the viewing plane to the second physical barrier and back again and pass through the irradiation space 2 in the process. In the present example, the irradiation space 2 comprises a total of four lighting means 11.1, 11.2 on each side of the panel and accordingly as an irradiation device 10. A person inside the irradiation space would be irradiated from both sides by these four panels. This system can also be provided with an auditory or other instruction output, which requires that the person passes through the irradiation space with appropriate gestures. Analogously, as described above, the sensor 21 can be used to record a thermal image of the person and to carry out a corresponding verification of the effectiveness of the application.

FIG. 2b then shows the previously described passage in the direction opposite of FIG. 2a. Correspondingly, a second input field 23 is provided at control station 7.3, through which a person can also pass with appropriate release means.

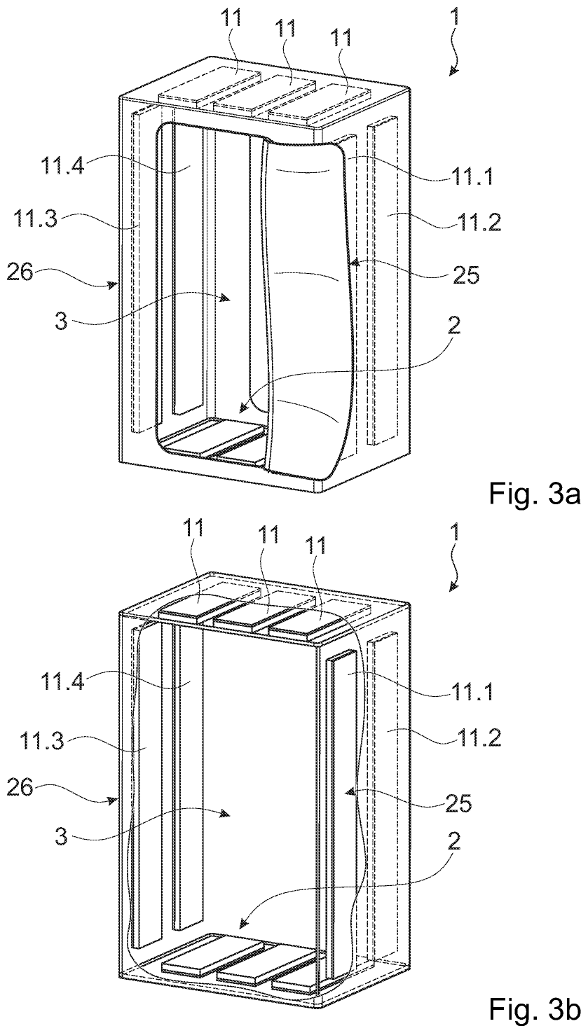

The embodiment of the device according to the invention shown in FIGS. 2a, 2b is especially suitable for protecting areas through which it is desired that people pass at a certain speed, such as in airports, shopping centers, or subway entrances. If there is a need to place a mobile device according to the present invention, this can be done with a device according to FIG. 3a. The device 1 according to the invention shown in FIG. 3a comprises a frame structure with a row of lighting means 11, 11.1, 11.2, 11.3, 11.4. The framework 26 is used to fasten a film 25. The film 25 can serve to define an irradiation space 2. In the present example, a zipper is provided with which a first physical barrier can be opened and the irradiation space 2 can thus be entered. The film can consist of a PVC plastic, an acrylic, or a polyester plastic. The material is preferably chosen so that it has high UV resistance.

During operation, a person would open the zipper of the film 25 and step into the irradiation space 2 formed by the framework 26. The person would then close the zipper again, so that a hermetic chamber is created. After a predetermined exposure to the lighting means 11F, the person would leave the irradiation chamber 2 again in a passage direction.

The internal structure of the device 1 from FIG. 3a is better illustrated in FIG. 3b, where parts of the film 25 are omitted and the corresponding elements are visible. The framework can serve to attach individual hooks and carrier wires (not shown) of the individual lighting means. A curtain or another zipper can be provided as a physical barrier at the exit, i.e. in the passage direction.

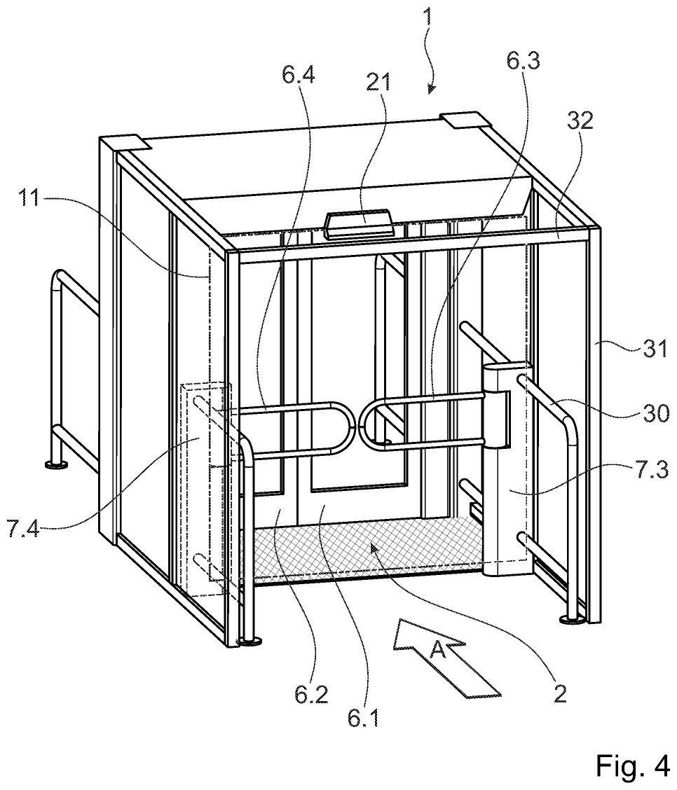

FIG. 4 again shows an embodiment of the device 1 according to the invention, which can be provided as a fixed installation. In this device as well, a physical barrier consists of an entry barrier in order to even enter the irradiation space 2, and an exit barrier in order to leave the irradiation space 2 in the passage direction A. The first physical barrier is a tree-style lock, which ensures that only one or at most two people can enter the irradiation space 2 at any one time. The second physical barrier is designed, analogously to FIG. 1a, 1b, as a pair of swing doors 6.1, 6.2. The first physical barrier is formed, analogously to FIG. 2a, 2b, from a pair of swing barriers 6.3, 6.4, which are actuated via control stations 7.3, 7.4. A corresponding framework 30 can be provided in order to anchor the physical barrier and to channel the people passing through. The irradiation space is also defined by a framework 31 with a ceiling beam 32. Above the irradiation space 2, there is a sensor 21 which detects when a person is in the irradiation space. Indicated laterally in the figure by dashed lines, irradiation devices 11 are arranged which, in the present case, irradiate a person from the side. These can be incorporated into wall panels or placed as corner pillars of the frame 31.

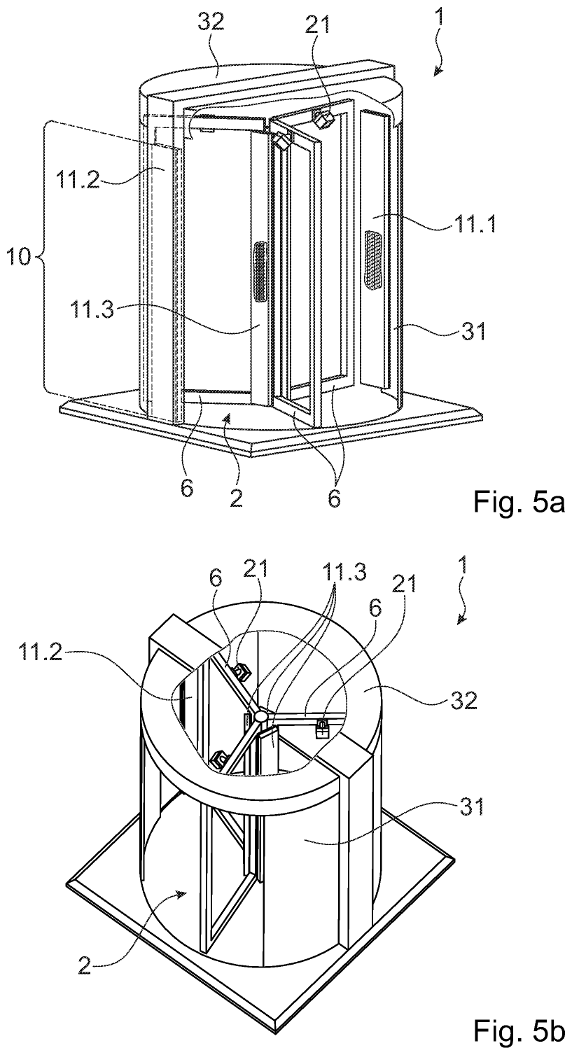

FIGS. 5a and 5b show a device 1 according to the invention, in which an additional isolation effect can be achieved with the access control. The device 1 is designed as a revolving door which is mounted on a pedestal, wherein the irradiation space 2 in each case forms a gusset of the revolving door arrangement. Each revolving door arrangement comprises a revolving door leaf 6 as well as an optical sensor 21 mounted on the revolving door leaf 6, which optical sensor 21 is arranged rotatably about a central axis and is accommodated in a device chamber 32. The inner radii of the individual gussets are each equipped with panels which comprise lighting means 11.3 which rotate with the entire revolving door arrangement. An irradiation device 10 is also provided on each side, permanently installed on a support frame at the outer radii, which in turn have lighting means 11.1, 11.2 and act on the irradiation space during use. The mode of operation is clearly visible in FIG. 5b, where the three lighting means 11.3 attached radially on the rotary element in the inner radius are visible.

Overall, such a device does in fact define three irradiation spaces 2, with each gusset of the revolving door forming its own irradiation space. Because the revolving doors 6 are largely made of glass, the laterally arranged lighting means 11.1, 11.2 also act in the distant gussets and irradiation spaces.

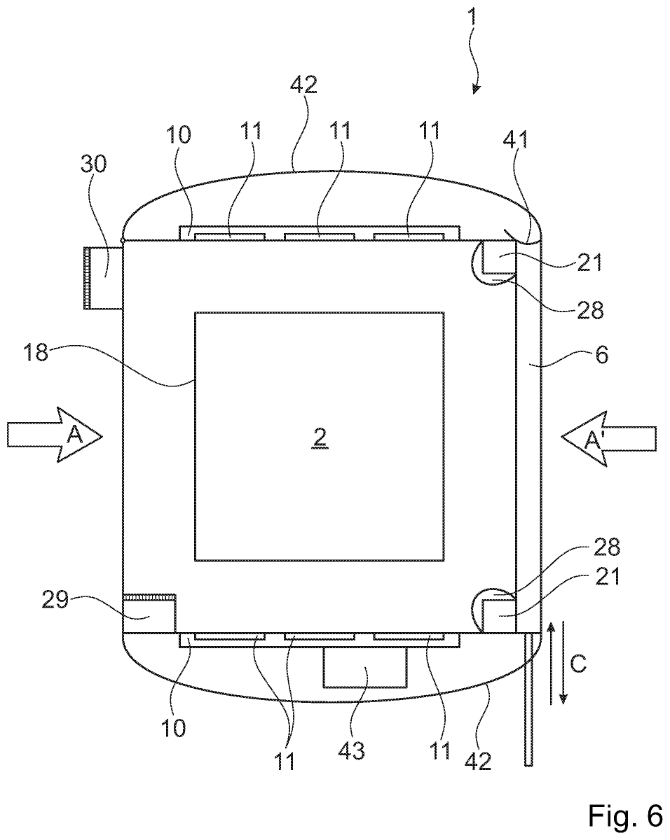

The mode of operation of the device according to the invention is illustrated schematically in FIG. 6. The device 1 has a passage direction A, which can of course also be traversed in the opposite direction as a reverse passage direction A′. A person who then wants to pass through this device 1 enters an irradiation space 2 in passage direction A, for example by having been identified at a control station 30 or by entering a corresponding release code at the control station 30. The person 2 in the irradiation space can then receive auditory information by means of a loudspeaker 29, which explains the disinfection process as a whole and, if necessary, explains whether certain postures are to be assumed. Optionally, a screen can be added to the loudspeaker 29, which graphically shows specific instructions accordingly. Further passage in the passage direction is blocked by a physical barrier. In the present example, the physical barrier is a sliding door 6 which can be moved in both directions C along a slide rail and can thus be transitioned from a closed state, as shown, to an open state. On both sides of the physical barrier, two panels 42 are provided, which comprise the irradiation device 10 and other electrical components. A control unit 43 can thus also be accommodated in these panels 42. If a person is then detected in the irradiation space by an optical sensor 21, the control unit 43 can run a corresponding exposure program which reaches a certain disinfection level. The optical sensors 21 can be accommodated in corresponding housings 28, which also protect the sensors from UV radiation or prevent manipulation of the sensors. If the disinfection is then concluded, the physical barrier opens and the person can continue to exit the device in passage direction A.

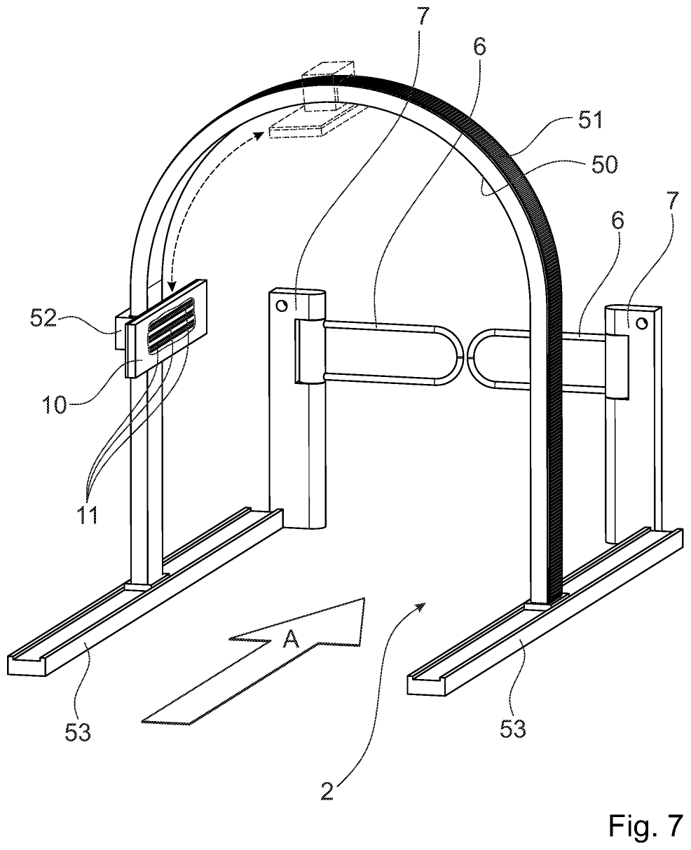

FIG. 7 describes a particular embodiment of the device according to the invention, which is especially suitable for mobile use. In this example, the physical barrier consists of a control station with two swing leaves 6. The physical barrier can be preinstalled or can also be easily operated manually, for example by unlocking and locking it by hand. To ensure disinfection, an irradiation device 10 is attached in front of the physical barrier in passage direction A. The irradiation device 10 is attached to a frame 50 which has a frame profile 51. The frame profile 21 can be traversed accordingly by a profile runner 52, which can be moved in a belt drive, so that the irradiation device 10 is mounted displaceably along the frame 50. The irradiation device 10 can thus define an irradiation space by defining a radius around an irradiation space. The frame 50 rests on two feet 53. The entire system can be mounted ad hoc in certain situations in order to provide a device for access control at short notice which includes physical disinfection.

The solution according to the invention provides a device of the type mentioned at the beginning, which can be used in a variety of ways, uses a safe and largely harmless technology for disinfecting skin surfaces or objects, and can be used in a modular manner in a wide range of applications.

1. A device (1) for access control, comprising
a. a first physical barrier (1) for restricting access to an irradiation space (2) along a passage direction (A);
b. at least one irradiation device (10) for exposing a living being (3) in the irradiation space (2) to optical radiation in a wavelength range of between 200 and 230 nm, in particular optical radiation with a peak in a wavelength range of between 207 and 222 nm.

2. The device according to claim 1, wherein the irradiation device comprises a lighting means that is excimer-based, in particular comprises a Kr-Br-excimer or a Kr—Cl-excimer lamp.

3. The device according to either of claim 1 or 2, comprising a first sensor, in particular an optical sensor.

4. The device according to claim 3, wherein the first sensor is an infrared sensor, in particular an infrared sensor, which is designed to record a thermal image of a living being in the irradiation space.

5. The device according to any of claims 1 to 4, wherein the physical barrier can be converted from a closed to an open state.

6. The device according to any of claims 1 to 5, comprising a control unit for actuating the physical barrier, and wherein the control unit is designed to be actuated by means of predefined criteria, in particular at least one predefined criterion from the group consisting of: length of stay of the living being in the irradiation space, body temperature of the living being, changes in the body temperature of the living being, exposure time of the living being to optical radiation in a wavelength range between 200 nm and 230 nm, exposure intensity of the living being to optical radiation in a wavelength range between 200 nm and 230 nm, changes in the surface temperature of the living being, the medical condition of the living being, and optical recognition of the living being.

7. The device according to any of claims 1 to 6, wherein the irradiation space is designed as an irradiation chamber and the physical barrier is designed to essentially hermetically seal off the irradiation space.

8. The device according to claim 7, wherein the physical barrier is transitioned from an open to a hermetically sealed state by actuation.

9. The device according to any of claims 1 to 8, comprising a ventilation unit for conveying an air flow into and/or out of the irradiation space.

10. The device according to claim 9, wherein the ventilation unit comprises a disinfection chamber which is designed to physically disinfect the air flow.

11. The device according to any of claims 1 to 10, wherein the physical barrier comprises at least one sliding door.

12. The device according to any of claims 1 to 11, comprising an emergency release for mechanically transitioning the physical barrier into an open state.

13. The device according to any of claims 1 to 12, comprising a second sensor for the optical detection of physiognomic properties for the purpose of face recognition.

14. The device according to any of claims 1 to 13, comprising a third sensor for detecting a living being along a passage direction (A) in front of the device.

15. The device according to any of claims 1 to 14, wherein the physical barrier is designed both to grant access to the irradiation space and to enable exiting the irradiation space.

16. The device according to any of claims 1 to 16, comprising a control panel for controlling a control unit.

17. The device according to any of claims 1 to 16, wherein irradiation device is arranged to be movable so that an irradiation area can be traversed.
The device according to any of claims 1 to 17, wherein the device comprises a plurality of irradiation spaces, wherein each irradiation space is formed as a module of an irradiation device for exposing a living being or parts of a living being to optical radiation in a wavelength range of between 200 and 230 nm, in particular optical radiation with a peak in a wavelength range of between 207 and 222 nm.

18. A method for access control, comprising the following steps:
a. Providing a device for access control, in particular according to claim 1, in a passage direction;
b. Transitioning a physical barrier of the device for access control from an open to a closed state as soon as a living being is in an irradiation space of the device for access control;
c. Exposure of the irradiation space to an irradiation device, which is designed to emit optical radiation in a wavelength range of between 200 and 230 nm, in particular optical radiation with a peak in a wavelength range of between 207 and 222 nm.

19. The method according to claim 19, further comprising the step of detecting at least one living being in the irradiation space by means of a sensor, in particular an optical sensor.

20. The method according to either of claim 19 or 20, further comprising the following steps:
a. Generating a thermal image of the living being before the start of exposure, in particular by means of an infrared sensor;
b. Continuous recording of a thermal image of the living being during exposure.

21. The method according to any of claims 19 to 21, wherein a control unit actuates a transition of the physical barrier from a closed to an open state by means of predefined criteria, in particular by means of at least one predefined criterion from the group consisting of: length of stay of the living being in the irradiation space, body temperature of the living being, changes in the body temperature of the living being, exposure time of the living being to optical radiation in a wavelength range between 200 nm and 230 nm, exposure intensity of the living being to optical radiation in a wavelength range between 200 nm and 230 nm, changes in the surface temperature of the living being, the medical condition of the living being, and optical recognition of the living being.

22. The method according to any of claims 19 to 22, wherein a length of stay in the irradiation space is defined in such a way that 90% of viruses and/or viroids are inactivated in the exposure area of the irradiation device in the irradiation space.

23. The method according to any of claims 19 to 23, wherein a length of stay is determined on the basis of data measured by sensors.

24. The method according to any of claims 19 to 24, wherein a difference in the surface temperature of the living being is determined, and the length of stay of the living being in the irradiation space, in particular in the exposure area of the irradiation unit, is determined on the basis of this difference.

25. A use of an irradiation means which is designed to emit optical radiation in a wavelength range of between 200 and 230 nm, for application to an irradiation space of a device (1) for access control, wherein the device comprises a physical barrier for restricting access to an irradiation space (2) along a passage direction.
